(19) United States
(12) Patent Application Publication
(10) Pub. No.: US 2014/0341818 A1
(43) Pub. Date: Nov. 20, 2014

(54) SOLUBILIZED MAGNOLOL ANALOGS

(71) Applicants: Regina Hourigan, Metuchen, NJ (US); Jeffrey Mastrull, Flemington, NJ (US); Jairajh Mattai, Piscataway, NJ (US); James Masters, Ringoes, NJ (US)

(72) Inventors: Regina Hourigan, Metuchen, NJ (US); Jeffrey Mastrull, Flemington, NJ (US); Jairajh Mattai, Piscataway, NJ (US); James Masters, Ringoes, NJ (US)

(73) Assignee: COLGATE-PALMOLIVE COMPANY, New York, NY (US)

(21) Appl. No.: 14/362,885

(22) PCT Filed: Dec. 15, 2011
(86) PCT No.: PCT/US2011/065016

§ 371 Date: Jun. 4, 2014

(51) Int. Cl. A61K 8/34; A61Q 19/10; A61Q 11/00; A61K 8/49

(52) U.S. Cl. CPC A61K 8/347; A61K 8/4973; A61Q 19/10; A61Q 11/00; A61K 2800/74; A61K 2800/49
USPC 424/49; 514/733

(57) ABSTRACT

A composition comprising a solubilized magnolol analog comprising at least one magnolol analog chosen from propyl magnolol, isopropyl magnolol, butyl magnolol, and isobutyl magnolol, and dimethyl isosorbide. These solubilized analogs are useful in personal care, oral care, and home care compositions to provide anti-bacterial activity and reducing the expression of pro-inflammatory mediators.

### FIELD OF THE INVENTION

Disclosed are solubilized magnolol analogs.

### BACKGROUND OF THE INVENTION

Magnolol analogs, such a propyl magnolol, isopropyl magnolol, butyl magnolol, and isobutyl magnolol, are known to have anti-bacterial activities and they are also shown to be capable of reducing the expression of pro-inflammatory mediators in oral tissues. The problem with using these magnolol analogs is their solubility in typical personal care, oral care, or home care compositions. Their use has been limited by their solubility. It would be desirable to solubilize these analogs to increase their use in personal, oral, or home care compositions. The problem is finding materials that can solubilize these analogs. Even in a given class of material, not all members of the class are effective at solubilizing these analogs.

### BRIEF SUMMARY OF THE INVENTION

A composition comprising a solubilized magnolol analog comprising at least one magnolol analog chosen from propyl magnolol, isopropyl magnolol, butyl magnolol, and isobutyl magnolol, and dimethyl isosorbide.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE INVENTION

The following description of the preferred embodiment(s) is merely exemplary in nature and is in no way intended to limit the invention, its application, or uses.

Disclosed is a composition comprising a solubilized magnolol analog comprising at least one magnolol analog chosen from propyl magnolol, isopropyl magnolol, butyl magnolol, and isobutyl magnolol, and dimethyl isosorbide.

Propyl magnolol is 5,5′-di-n-propylbiphenyl-2,2′-diol, butyl magnolol is 5,5′-di-n-butylbiphenyl-2,2′-diol, isopropyl magnolol is 5,5′-di-isopropylbiphenyl-2,2′-diol, and isobutyl magnolol is 5,5′-di-isobutylbiphenyl-2,2′-diol.

The dimethyl isosorbide is capable of solubilizing up to 200 g per liter of neat propyl magnolol or isopropyl magnolol, up to 100 g per liter of isobutyl magnolol, or up to 50 g per liter of butyl magnolol. In certain embodiments, the amount of dimethyl isosorbide is at least 5 times the weight of the propyl magnolol or isopropyl magnolol in the composition. In certain embodiments, the amount of dimethyl isosorbide is at least 10 times the weight of isobutyl magnolol in the composition. In certain embodiments, the amount of dimethyl isosorbide is at least 20 times the weight of butyl magnolol in the composition.

Dimethyl isosorbide is available as Arlasove™ DMI from Croda.

It was surprising that dimethyl isosorbide was able to solubilize these analogs. Many other solubilizers, such as PEG-7 glyceryl cocoate, poloxamer 124, PPG-2 hydroxyethyl cocoamide, PPG-5 laureth-5 (Eumulgin™ ES), PEG-8/SMDI copolymer, isopropyl myristate, or C12-15 alkyl benzoate are not able to solubilize isobutyl magnolol.

The amount of magnolol analog in the composition can be any desired amount. In certain embodiments, the amount is 0.01 to 5% by weight of the composition. In other embodiments, the amount is at least 0.05, at least 0.1, at least 0.2, at least 0.3, at least 0.4, at least 0.5, or at least 1% by weight up to 5% by weight of the composition. In other embodiments, the amount is any of the foregoing minimum amounts up to 4, up to 3, up to 2, or up to 1% by weight of the composition. The weight of the dimethyl isosorbide is then the amount to solubilize the analog with the minimum amount of the dimethyl isosorbide being based on the maximum solubility of the analog in the dimethyl isosorbide. In certain embodiments, the amount of the magnolol analog is 0.1, 0.2, 0.3, 0.4, or 0.5% by weight.

These solubilized analogs are useful in personal care, oral care, and home care compositions. Examples of personal care compositions include, but are not limited to, body wash/shower gel, liquid hand cleanser, bar soap, shampoo, conditioner, antiperspirant/deodorants, and cosmetics. Examples of oral care compositions include, but are not limited to, dentifrices, toothpastes, tooth powders, prophylaxis pastes, mouth rinses, lozenges, gums, gels, paints, confectionaries, and denture cleaners. Examples of oral care compositions that can include solubilized magnolol analogs can be found in WO2011/106492. Examples of home care compositions include, but are not limited to, dish liquids, dish pastes, hard surface cleaners, fabric conditioners, and laundry detergents.

In certain embodiments, the magnolol analog can be present in a body wash/shower gel, liquid hand cleanser, or shampoo in which each of these compositions include a surfactant. The magnolol analog can also be included in a soap (fatty acid soap), which can be in the shape of a bar soap.

### EXAMPLES

The following are non-limiting prophetic examples of compositions that can include solubilized magnolol analogs.

### Liquid Cleanser (Body Wash or Liquid Hand Soap)

| | % Wt. | % Wt. | % Wt. |
|---|---|---|---|
| Ingredient Name | Range | Range | Range |
| Propyl magnolol or isopropyl | 0.01-1% | 0 | 0 |
| magnolol | | | |
| Butyl magnolol | 0 | 0.01-1% | 0 |
| Isobutyl magnolol | 0 | 0 | 0.01-1% |
| Dimethyl isosorbide | At least 5 | At least 10 | At least 20 |
| | times the | times the | times the |
| | weight of the | weight of the | weight of the |
| | magnolol | magnolol | magnolol |
| | analog | analog | analog |
| Polyquaternium-7 | 0-0.25 | 0-0.25 | 0-0.25 |
| SO₃Na Pareth 145-2EO | 8-12 | 8-12 | 8-12 |
| Sulfate | | | |
| Cocamidopropyl Betaine | 2.5-7 | 2.5-7 | 2.5-7 |
| Decyl Glucoside | 0-2 | 0-2 | 0-2 |
| Demineralized Water and | Q.S. | Q.S. | Q.S. |
| minors | | | |
| Total Materials | 100 | 100 | 100 |

### Bar Soap

| | % Wt. | % Wt. | % Wt. |
|---|---|---|---|
| Ingredient Name | Range | Range | Range |
| Propyl magnolol or isopropyl | 0.01-1% | 0 | 0 |
| magnolol | | | |
| Butyl magnolol | 0 | 0.01-1% | 0 |
| Isobutyl magnolol | 0 | 0 | 0.01-1% |
| Dimethyl isosorbide | At least 5 | At least 10 | At least 20 |
| | times the | times the | times the |
| | weight of the | weight of the | weight of the |
| | magnolol | magnolol | magnolol |
| | analog | analog | analog |
| Fatty acid soap | 75-85 | 75-85 | 75-85 |
| Demineralized Water and | Q.S. | Q.S. | Q.S. |
| minors | | | |
| Total Materials | 100 | 100 | 100 |

### Oral Care Composition

| Ingredient | Weight % | Weight % | Weight % |
|---|---|---|---|
| Purified water | Q.S. | Q.S. | Q.S. |
| Sorbitol | 19.45 | 19.45 | 19.45 |
| Glycerin | 20 | 20 | 20 |
| Sodium CMC-12 type USP | 1.1 | 1.1 | 1.1 |
| lota carrageenan (LB 9505) | 0.4 | 0.4 | 0.4 |
| Sodium saccharin-USP | 0.3 | 0.3 | 0.3 |
| Sodium fluoride | 0.24 | 0.24 | 0.24 |
| Zeodent-115-dental type | 8.5 | 8.5 | 8.5 |
| silica abrasive | | | |
| Zeodent-165-synthetic | 3 | 3 | 3 |
| amorphous PPT silica | | | |
| Dental type silica sylodent | 10 | 10 | 10 |
| XWA650 | | | |
| Titannium dioxide (TiO2) | 0.5 | 0.5 | 0.5 |
| Sodium lauryl sulphate | 1.5 | 1.5 | 1.5 |
| powder-NF | | | |
| Flavor | 1 | 1 | 1 |
| Propyl magnolol or isopropyl | 0.01-1% | 0 | 0 |
| magnolol | | | |
| Butyl magnolol | 0 | 0.01-1% | 0 |
| Isobutyl magnolol | 0 | 0 | 0.01-1% |
| Dimethyl isosorbide | At least 5 | At least 10 | At least 20 |
| | times the | times the | times the |
| | weight of the | weight of the | weight of the |
| | magnolol | magnolol | magnolol |
| | analog | analog | analog |
| Total | 100 | 100 | 100 |

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

1. A composition comprising a solubilized magnolol analog comprising:
at least one magnolol analog chosen from propyl magnolol, isopropyl magnolol, butyl magnolol, and isobutyl magnolol, and dimethyl isosorbide.

2. The composition of claim 1, wherein the magnolol analog is present in an amount of 0.01 to 5% by weight of the composition.

3. The composition of claim 1, wherein the dimethyl isosorbide is present in an amount of at least 5 times the weight of isopropyl magnolol or propyl magnolol, the dimethyl isosorbide is present in an amount of at least 10 times the weight of isobutyl magnolol, or the dimethyl isosorbide is present in an amount that is 20 times the weight of butyl magnolol.

4. The composition of claim 1 claim further comprising a surfactant.

5. The composition of claim 1 claim further comprising soap.

6. The composition of claim 5 in the form of a bar soap.

7. The composition of claim 6 in the form of a liquid cleanser.

8. The composition of claim 1 in the form of an oral care composition.
